(19) United States
(12) Patent Application Publication
(10) Pub. No.: US 2015/0224066 A1
(43) Pub. Date: Aug. 13, 2015

(54) ANTIMICROBIAL COMPOSITIONS AND METHODS OF USE

(71) Applicants: Patrick SCHLIEVERT, (US); UNIVERSITY OF IOWA RESEARCH FOUNDATION, Iowa City, IA (US)

(72) Inventor: Patrick Schlievert, Iowa City, IA (US)

(73) Assignee: UNIVERSITY OF IOWA RESEARCH FOUNDATION, Iowa City, IA (US)

(21) Appl. No.: 14/428,442

(22) PCT Filed: Mar. 14, 2013
(86) PCT No.: PCT/US13/31713

§ 371 Date: Mar. 16, 2015

Related U.S. Application Data
(60) Provisional application No. 61/705,553, filed on Sep. 25, 2012.

(51) Int. Cl. A61K 31/122; A61K 47/38; A61K 47/10; A61K 47/14

(52) U.S. Cl. CPC A61K 31/122; A61K 47/14; A61K 47/38; A61K 47/10

(57) ABSTRACT

The disclosure relates to antibiotic compositions and methods of use thereof in a mammal. In certain embodiments, the present invention provides an antimicrobial composition comprising a non-aqueous carrier and an active agent, wherein the active agent is menadione, 1,4-naphthoquinone, Coenzyme Q1, Coenzyme Q2, Coenzyme Q3, Coenzyme Q4, Coenzyme Q5, Coenzyme Q6, Coenzyme Q7, Coenzyme Q8, Coenzyme Q9, and/or Coenzyme Q10, or a pharmaceutically acceptable salt thereof.

### RELATED APPLICATIONS

This patent application claims the benefit of priority of U.S. Application Ser. No. 61/705,553, filed Sep. 25, 2012, which application is incorporated by reference herein.

### FEDERAL GRANT SUPPORT

This invention was made with government support under Grant No. AI074283 and AI57153 awarded by the National Institutes of Health. The government has certain rights in the invention.

### BACKGROUND OF THE INVENTION

Staphylococcus aureus is a gram-positive bacterium that causes large numbers of infections throughout the world. The organism is ubiquitous, with estimates of 30-40% of humans being colonized on mucosal surfaces (1,2). Illnesses caused by the organism range from benign infections, such as furuncles, to life-threatening illnesses such as toxic shock syndrome (TSS) (1-3). S. aureus causes diseases through production of cell surface and secreted virulence factors (1-4). One of the major secreted exotoxins is the superantigen TSS toxin-1 (TSST-1) (5-7). TSST-1 is the cause of menstrual TSS, an illness that may be seen in healthy women who are using tampons and who most often have vaginal colonization with S. aureus (8,9). Additionally, TSST-1 is the cause of up to 50% of non-menstrual TSS, with many cases associated with upper respiratory tract infections; most of the rest of non-menstrual TSS is associated with the superantigens staphylococcal enterotoxins B and C (8,9). Superantigens cause serious illnesses primarily through over-production of cytokines, resulting in acute-onset TSS illnesses with fever, vomiting and diarrhea, hypotension, a sunburn-like rash, peeling of the skin upon recovery, and a variable multi-organ component (2,10-12).

Streptococcus pyogenes and Streptococcus agalactiae are also gram-positive cocci that cause TSS in part through over-stimulation of cytokine production through superantigens and other factors (2,11,13-16). Additionally, these organisms cause other types of infections, including pharyngitis and impetigo as caused by Streptococcus pyogenes (17), and neonatal sepsis and meningitis as caused by Streptococcus agalactiae (18).

The gram-positive rod Bacillus anthracia depends on production of its cell surface capsule and other molecules and exotoxins to cause serious illnesses, including skin, gastrointestinal, and pulmonary anthrax (19,20). This organism is a category A select agent and consequently is considered a significant bioterrorism threat.

Currently, there is a need for improved treatments to prevent or ameliorate microbial infections in patients.

### SUMMARY OF THE INVENTION

In certain embodiments, the present invention provides an antimicrobial composition comprising a non-aqueous carrier and an active agent, wherein the active agent is menadione, 1,4-naphthoquinone, Coenzyme Q1, Coenzyme Q2, Coenzyme Q3, Coenzyme Q4, Coenzyme Q5, Coenzyme Q6, Coenzyme Q7, Coenzyme Q8, Coenzyme Q9, and/or Coenzyme Q10, or a pharmaceutically acceptable salt thereof.

In certain embodiments, the present invention provides an apparatus comprising a solid substrate and the antimicrobial composition described above.

In certain embodiments, the present invention provides a method of inhibiting growth of a microbe comprising administering to an animal in need thereof the composition described above.

In certain embodiments, the present invention provides a method of inhibiting production of an exotoxin from a microbe comprising administering to an animal in need thereof the composition described above.

In certain embodiments, the present invention provides inhibiting inflammation in an animal in need thereof comprising administering the composition described above.

The invention also provides a antimicrobial composition for use in medical therapy.

The invention also provides the composition described above for the prophylactic or therapeutic treatment of a microbial infection. In certain embodiments, the microbe is a gram-positive bacterium, a gram-negative bacterium, an enveloped virus, or a fungus. In certain embodiments, the microbe is a gram-positive bacterium Staphylococci aureus, Streptococci pyogenes, Streptococci agalactae, or Bacillus anthracis. In certain embodiments, the microbe is a gram-negative bacterium. In certain embodiments, the microbe is a gram-negative bacterium Gardnerella vaginalis. In certain embodiments, the microbe is an enveloped virus. In certain embodiments, the microbe is HIV or HCV. In certain embodiments, the microbe is a fungus, such as Candida albicans.

The invention also provides the use of the composition described above to prepare a medicament for treating a microbial infection in an animal (e.g. a mammal such as a human).

### BRIEF DESCRIPTION OF DRAWINGS

FIG. 1. Chemical structures of menaquinone and analogs.

FIG. 2. Chemical structures of coenzyme Q molecules.

FIGS. 3A-3D. Menaquinone does not inhibit the growth of S. aureus MN8 (A), B. anthracis Sterne (B), Streptococcus pyogenes MNWA (C), and Streptococcus agalactiae MNSI (D). Organisms were inoculated at designated CFUs/ml as indicated by the horizontal line (107 CFUs/ml for S. aureus and 106 CFUs/ml for other organisms). Final CFUs/ml were determined after 24 h incubation with indicated concentrations of menaquinone. Bars indicate standard deviations.

FIGS. 4A-4D. Menadione inhibits the growth of S. aureus MN8 (A), B. anthracis Sterne (B), Streptococcus pyogenes MNWA (C), and Streptococcus agalactiae MNSI (D). Organisms were inoculated at designated CFUs/ml as indicated by the horizontal line (10⁷ CFUs/ml for S. aureus and 10⁶ CFUs/ml for other organisms). Final CFUs/ml were determined after 24 h incubation with indicated concentrations of menadione. The lower limit of detection of organisms was 10 CFUs/ml. Bars indicate standard deviations.

FIG. 5. Coenzyme Q1-3 (CoQ1-3) but not coenzyme Q10 (CoQ10) inhibit the growth of S. aureus MN8. Organisms were inoculated at designated CFUs/ml as indicated by the horizontal line (10⁷ CFUs/ml). Final CFUs/ml were determined after 24 h incubation with indicated concentrations of CoQ1-3. The lower limit of detection or organisms was 10 CFUs/ml. Bars indicate standard deviations.

FIG. 6. Time-course for coenzyme Q1 (50 μg/ml) to inhibit the growth of S. aureus MN8. Organisms were inoculated at 10⁷ CFUs/ml. Final CFUs/ml were determined at designated time points for up to 24 h. The lower limit of detection of organisms was 10 CFUs/ml. (Open squares no coenzyme Q1) (Filled squares with coenzyme Q1).

FIGS. 7A-7B. Coenzyme Q1 (CoQ1) inhibits production of TSST-1 at CoQ1 concentrations that do not inhibit S. aureus MN8 growth. S. aureus MN8 (10⁷/ml) was cultured overnight with designated concentrations of CoQ1. Subsequently, CFUs/ml (A; open circles) and TSST-1 μg/ml (A; closed circles, B) were determined.

FIG. 8. Coenzyme Q1-3 (CoQ1-3) but not coenzyme Q10 (CoQ10) inhibit the growth of B. anthracis Sterne. Organisms were inoculated at designated CFUs/ml as indicated by the horizontal line (10⁶ CFUs/ml). Final CFUs/ml were determined after 24 h incubation with indicated concentrations of coenzyme Q1-3. The lower limit of detection of organisms was 10 CFUs/ml. Bars indicate standard deviations.

FIGS. 9A-9B. Coenzyme Q1 (CoQ1) but not coenzyme Q10 (CoQ10) inhibits the growth of Streptococcus pyogenes MNWA (A) and Streptococcus agalactiae MNSI (B). Organisms were inoculated at designated CFUs/ml as indicated by the horizontal line (10⁶ CFUs/ml). Final CFUs/ml were determined after 24 h incubation with indicated concentrations of coenzyme Q1. The lower limit of detection of organisms was 10 CFUs/ml. Bars indicate standard deviations.

FIG. 10. Menadione and coenzymeQ1 (CoQ1) are more effective at inhibiting the growth of S. aureus MN8 when cultured aerobically (filled squares) compared to culturing anaerobically (open squares). The lower limit of detection of organisms was 10 CFUs/ml.

FIG. 11. Glycerol monolaurate (GML) synergizes with CoQ1 to kill S. aureus MN8 in a 24 h test period. S. aureus MN8 (10⁷/ml) was incubated in TH broth with varying concentrations of CoQ1 or varying concentrations of CoQ1+GML (10 μg/ml). GML (100 μg/ml) was not inhibitory to S. aureus.

FIG. 12. Coenzyme Q1 (CoQ1) protects rabbits from TSS in a subcutaneous abscess model. Rabbits received subcutaneous Wiffle balls 6 weeks prior to experimentation for encapsulation of the Wiffle balls. The animals were then injected intra-Wiffle ball with S. aureus MN8+CoQ1 in ethanol or S. aureus MN8+ethanol. Animals were monitored for survival over a one week test period. P value for survival difference was determined by Fisher's Exact test.

FIG. 13. Growth charts for menaquinone analog CoQ1 at concentrations of (0-50 μg/ml) against Gardnerella vaginalis and Candida albicans.

FIG. 14. Test of CoQ1 for ability to prevent development of TSS induced by S. aureus in rabbits in a subcutaneous model.

### DETAILED DESCRIPTION OF THE INVENTION

Gram-positive two-component systems (TCS) have been identified that affect virulence and ability to interact with the external environment (21-23). One or more of these critical TCS are regulated by menaquinone or related molecules. Part of the basis for this is that many compounds tested, including hemoglobin (24), clindamycin (25), glycerol monolaurate (26), and chitosan (27), although disparate in properties, appear to target S. aureus and Streptococcus pyogenes bacterial plasma membrane TCS signaling, with the consequence being reduction of growth and/or exotoxin production. This suggests that menaquinone analogs also inhibit gram-positive bacterial growth and exotoxin production.

### Antimicrobial Compositions

In certain embodiments of the present invention, the active agent is menadione, 1,4-naphthoquinone, Coenzyme Q1, Coenzyme Q2, Coenzyme Q3, Coenzyme Q4, Coenzyme Q5, Coenzyme Q6, Coenzyme Q7, Coenzyme Q8, Coenzyme Q9, and/or Coenzyme Q10. In certain embodiments, the active agent is menadione or 1,4-naphthoquinone. In certain embodiments, the active agent is Coenzyme Q1, Coenzyme Q2, and/or Coenzyme Q3. In certain embodiments, the active agent is present at a concentration of about 3-500 μg/ml μg/ml. In certain embodiments, the active agent is present at a concentration of about 10-200 μg/ml. In certain embodiments, the active agent is present at a concentration of about 10-50 μg/ml.

In certain embodiments, the antimicrobial composition further comprises a glycerol monoester (GME). In certain embodiments, the GME is glycerol linked to a C6-C22 acyl group (e.g., C(═O)C5-C21 alkyl, wherein the alkyl is branched or unbranched, saturated or unsaturated). In certain embodiments, the acyl group is branched or unbranched, saturated or unsaturated. In certain embodiments, the acyl group is unbranched and saturated. In certain embodiments, the acyl group is derived from a fatty acid. In certain embodiments, the acyl group is derived from a saturated fatty acid, e.g., caprylic acid, capric acid, lauric acid, myristic acid, palmitic acid, stearic acid, arachidic acid, or behenic acid. In certain embodiments, the GME is glycerol monocaprylate (C8), glycerol monocaprate (C10), glycerol monolaurate (C12, “GML”), or glycerol monomyristate (C14). In certain embodiments, the GME is present at a concentration of about 10-10,000 μg/ml, or any value therebetween. In certain embodiments, the GME is GML. In certain embodiments, the composition comprises Coenzyme Q1 and GML.

In certain embodiments of the present invention, the carrier is one or more non-aqueous carriers or gels. In certain embodiments, the carrier is, for example, olive oil, vegetable oil, or a petroleum jelly.

In certain embodiments, the antimicrobial composition is at a pH of about 4.0-8.0. In certain embodiments the antimicrobial composition further comprises an accelerant. In certain embodiments, the accelerant is EDTA. In certain embodiments, the antimicrobial composition comprises, consists essentially of or consists of Propylene glycol (Gallipot, St. Paul, Minn.) (73.55% v/v), polyethylene glycol (Gallipot) (25% v/v) and hydroxypropyl cellulose (Gallipot) as a gelling agent (1.25% w/v). In certain embodiments, the compounds are heated to 65° C. for solubilization of components and for solubilization of CoQs and GME, such as GML.

In certain embodiments of the present invention, the antibiotic composition is applied to a solid substrate, such as a medical device or a consumer product (e.g., a diaper). In certain embodiments, the active agent is dispersed in a polymer.

### Apparatuses

In certain embodiments, the present invention provides an apparatus comprising a solid substrate and the antimicrobial composition described above. In certain embodiments, the solid substrate is coated with the antimicrobial composition. In certain embodiments, solid substrate is a metal, fabric, or polymeric surface. In certain embodiments, solid substrate is a biostable or bioabsorbable composition.

### Methods of Use

In certain embodiments, the present invention provides a method of inhibiting growth of a microbe comprising administering to an animal in need thereof a composition described above.

In certain embodiments, the present invention provides a method of inhibiting production of an exotoxin from a microbe comprising administering to an animal in need thereof a composition described above.

In certain embodiments, the present invention provides a method of inhibiting inflammation in an animal in need thereof comprising administering a composition described above.

In certain embodiments, the microbe is a gram-positive bacterium, a gram-negative bacterium, an enveloped virus, or a fungus. In certain embodiments, the microbe is a gram-positive bacterium Staphylococci aureus, Streptococci pyogenes, Streptococci agalactae, or Bacillus anthracis. In certain embodiments, the microbe is Staphylococci aureus and the composition comprises Coenzyme Q1 and GML. In certain embodiments, the microbe is a gram-negative bacterium Gardnerella vaginalis.

In certain embodiments, the microbe is an enveloped virus. In certain embodiments, the virus is HIV or HCV.

In certain embodiments, the microbe is a fungus. In certain embodiments, the fungus is Candida albicans.

### Methods of Administration

The active agents of the present invention can be formulated as pharmaceutical compositions and administered to a mammalian host, such as a human patient in a variety of forms adapted to the chosen route of administration, i.e., orally or parenterally, by intravenous, intramuscular, topical or subcutaneous routes.

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds of formula I to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compounds of formula I can be determined by comparing their in vitro activity, and in vivo activity in animal models. Methods for the extrapolation of effective dosages in mice, and other animals, to humans are known to the art; for example, see U.S. Pat. No. 4,938,949.

The amount of the compound, or an active salt or derivative thereof, required for use in treatment will vary not only with the particular agent selected but also with the route of administration, the nature of the condition being treated and the age and condition of the patient and will be ultimately at the discretion of the attendant physician or clinician.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

Compounds of the invention can also be administered in combination with other therapeutic agents, for example, other agents that are useful as antibiotics or anti-inflammatory agents. Accordingly, in one embodiment the invention also provides a composition comprising an active agent of the present invention and at least one other therapeutic agent, and a pharmaceutically acceptable diluent or carrier. The invention also provides a kit comprising a compound of formula I, or a pharmaceutically acceptable salt thereof, at least one other therapeutic agent, packaging material, and instructions for administering the compound of formula I or the pharmaceutically acceptable salt thereof and the other therapeutic agent or agents to an animal to treat or prevent a microbial infection.

The invention will now be illustrated by the following non-limiting Examples.

### Example 1

### Novel Antimicrobial Agents Inhibit Microbes

Many gram-positive bacteria cause serious human illnesses through combinations of cell-surface virulence factors and production of exotoxins. We initiated studies with four of these organisms to develop novel therapeutic agents that interfere with growth, focusing on menaquinone analogues. Multiple compounds were identified (menadione, 1,4-naphthoquinone, and coenzyme Q1-3 but not menaquinone, phylloquinone, and coenzyme Q10) which were effective at inhibition of growth and to a greater extent exotoxin production in vitro for Staphylococcus aureus, Bacillus anthracis, Streptococcus pyogenes, and Streptococcus agalactiae at concentrations of 10-200 μg/ml. Coenzyme Q1 synergized with another antimicrobial agent, glycerol monolaurate, to inhibit S. aureus growth. Coenzyme Q1, was non-toxic to rabbits as assessed by lack of gross pathology, was antimicrobial, and reduced the ability of S. aureus to cause toxic shock syndrome in a rabbit abscess model. Menaquinone analogs both induce toxic reactive oxygen species and affect bacterial plasma membranes and biosynthetic machinery to interfere with critical two-component signal transduction systems, respiration, and macromolecular synthesis. These compounds represent a novel class of useful therapeutic agents.

In recent studies, we and others have identified gram-positive two-component systems (TCS) that affect virulence. We hypothesize that one or more of these TCS may be regulated by menaquinone or related molecules. Part of the basis for this hypothesis is that many compounds we have tested, including hemoglobin, clindamycin, glycerol monolaurate, and chitosan, although disparate in properties, appear to target S. aureus and Streptococcus pyogenes bacterial plasma membrane TCS signaling, with the consequence being reduction of growth and/or exotoxin production. This suggests that it may be possible to find menaquinone analogs that also inhibit gram-positive bacterial growth and exotoxin production, forming the basis for our studies.

Materials and Methods

Bacteria.

S. aureus MN8, a typical menstrual TSS isolate representing approximately 75% of such isolates, was used in many of our studies (7). The organism is maintained in the Schlievert laboratory in the lyophilized state. Two other S. aureus strains were also studied in some experiments. These are CDC587, a typical menstrual TSS isolate (7) and MNPE a non-menstrual TSS isolate associated with fatal post-influenza pneumonia (28). Bacillus anthracis Sterne was purchased from the Colorado Serum Company, Denver, Colo. For experimentation, S. aureus MN8, CDC587, and MNPE, and B. anthracis Sterne were cultured overnight in Todd Hewitt (TH) broth (Difco Laboratories, Detroit, Mich.). The next day, the organisms were diluted in fresh TH broth to 10⁶ to 10⁷ colony-forming units (CFUs)/ml for inoculation. Streptococcus pyogenes strain MNWA is an M3 isolate from a TSS patient. Streptococcus agalactiae strain MNSI is an isolate from a patient with neonatal sepsis. These organisms are also maintained in the Schlievert laboratory in the lyophilized state. The organisms were cultured stationary in TH broth in the presence of 7% CO₂. On the day of experimentation, the organisms were diluted to approximately 10⁶ CFU/ml for inoculation.

Menaquinone Derivatives.

Menaquinone and derivatives (menadione, 1,4-naphthoquinone, phylloquinone, coenzyme Q1-3, and coenzyme Q10) were purchased from Sigma-Aldrich, St. Louis, Mo. The compounds were studied in dose response experiments with concentrations ranging from 0-200 μg/ml.

Menaquinone Analog Effects on S. aureus and TSST-1 Production.

For aerobic studies of S. aureus, strain MN8, CDC587, and MNPE were cultured in 25 ml TH for 1-24 h with shaking (200 revolutions/min) in the presence and absence of potential antimicrobial compounds. After incubation, a sample of each culture was used for plate-count determination of CFUs/ml, and a sample was used for TSST-1 quantification by Western immunoblot analysis (24). For TSST-1 determination, 1 ml of each sample was treated overnight with 4 volumes of absolute ethanol; we have previously shown that this treatment precipitates all measurable TSST-1 (25). Subsequently, the precipitate from each culture was collected by centrifugation (4000×g, 10 min), ethanol poured off, and sample dried for 30 min under a laminar flow hood. Each sample was resuspended in distilled water (100 μl) and clarified by centrifugation (14,000×g, 5 min). Ten microliters of clarified sample was added to 10 μl of sodium-dodecyl sulfate polyacrylamide gel electrophoresis (SDS-PAGE) sample buffer (final volume 1/10^{th} original culture volume). The total of 20 μl sample+buffer was electrophoresed on SDS-PAGE gels and transblotted onto 0.2 μm PVDF membranes; control samples of purified TSST-1 were treated comparably, ranging from 10 μg/lane to 0.01 μg/lane. Subsequently, Western immunoblots were developed with hyperimmune antibodies against TSST-1 (Toxin Technologies, Sarasota, Fla.), then alkaline phosphatase-conjugated anti-rabbit IgG (Sigma-Aldrich), and finally substrate. The color reactions were visualized by using NIH program ImageJ for quantitative comparison to purified TSST-1 samples (24). The standard curve generated from purified TSST-1 typically gave R² values of greater than 0.95, consistent with the reliability of this technique to be used quantitatively (24).

For anaerobic chamber experiments, S. aureus MN8 was cultured stationary overnight in TH broth that had been incubated in an anaerobic chamber for two days. Then, the organism was inoculated into 1 ml volumes of anaerobic TH broth in the chamber. The cultures were incubated stationary in the presence of various concentrations of antimicrobial agents for 24 h, and then assayed for CFUs/ml by plated counting.

Menaquinone Analog Effects on B. anthracis, Streptococcus Pyogenes, and Streptococcus agalactiae Growth.

Organisms were inoculated into 25 ml TH and incubated for up to 24 h with antimicrobial compounds. B. anthracis Sterne was cultured at 37° C. with shaking (100 revolutions/min) aerobically. Streptococci were also inoculated into TH and incubated with antimicrobial compounds for up to 24 h, but these organisms were grown stationary in the presence of 7% CO₂.

Glycerol Monolaurate-CoQ1 Synergy Study.

Varying concentrations of CoQ1 and varying concentrations of CoQ+GML (10 μg/ml) were added to TH flasks with S. aureus MN8 (10⁷/ml). An additional set of flasks contained GML alone at 10 μg/ml and 100 μg/ml. All flasks were incubated aerobically with shaking (200 RPM) for 24 h. Subsequently, CFUs/ml in triplicate were determined.

Rabbit Model for TSS.

All experiments were performed under an approved IACUC protocol. A subcutaneous abscess model was used to assess the ability of coenzyme Q1 to prevent TSS. Briefly, Wiffle golf balls were surgically implanted subcutaneously in rabbit flanks, one ball/animal. The rabbits, either sex, were allowed to heal and encapsulate the golf balls for 6 weeks. Then, 1.5 ml of the 30 ml serous fluid within the golf balls was removed and replaced with coenzyme Q1 (0.5 mg in 0.5 ml ethanol) or vehicle (0.5 ml ethanol) plus S. aureus MN8 (5×10⁸ in a 1.0 ml volume). Rabbits were monitored for up to 1 week for development of lethal TSS. Rabbits were prematurely euthanized when they simultaneously could not right themselves and failed to exhibit escape behavior. This point has been shown experimentally to be 100% predictive of lethal TSS.

Results

Menaquinone and Analog Structures.

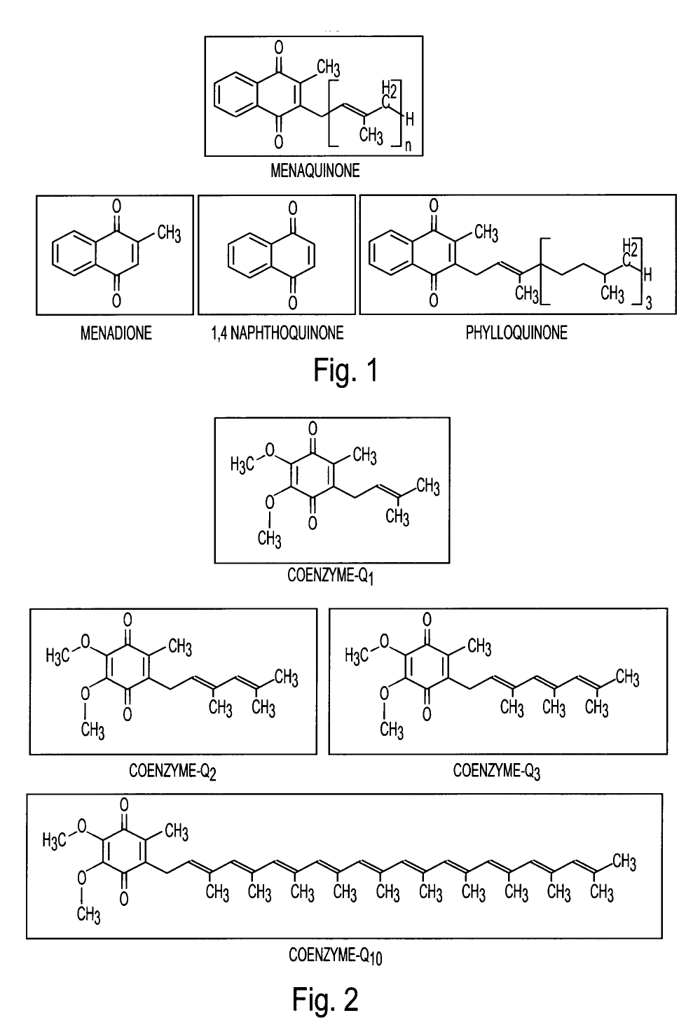

The structures of menaquinone, menadione, 1,4-naphthoquinone, and phylloquinone that were tested for antimicrobial activity and inhibition of exotoxin production are shown in FIG. 1. We also tested 4 coenzyme Q family members, coenzyme Q1-3 and coenzyme Q10, a health-food supplement (FIG. 2). The difference among the coenzyme Q molecules is the number of isoprenyl units in the side-chains.

Menaquinone and Phylloquinone do not Inhibit Bacterial Growth.

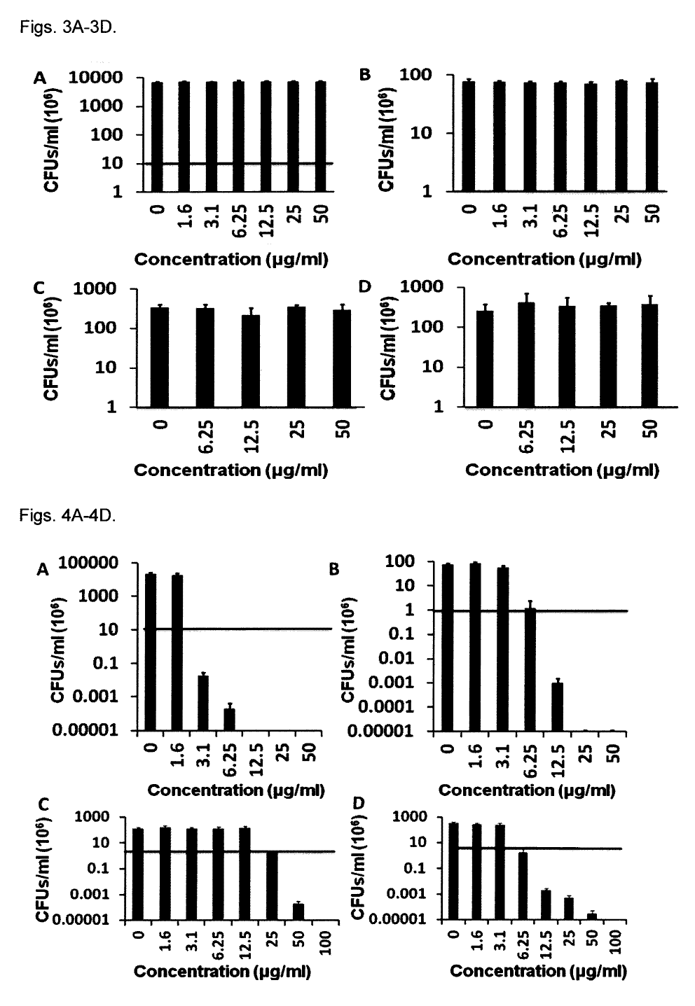

Menaquinone as expected lacked antimicrobial activity at any concentration tested (0-50 μg/ml) against S. aureus MN8 (FIG. 3A), B. anthracis Sterne (FIG. 3B), Streptococcus pyogenes MNWA (FIG. 3C), and Streptococcus agalactiae MNSI (FIG. 3D). Phylloquinone also exhibited no antimicrobial activity against any of the four organisms. We tested menaquinone for antimicrobial activity against two other TSS S. aureus strains: CDC587, MNPE. Menaquinone showed no antimicrobial activity against either of these two strains. Menaquinone and phylloquinone also did not inhibit production of TSST-1 by S. aureus MN8, CDC587, and MNPE at any drug concentration compared to the no drug controls.

Menadione and 1,4-Naphthoquinone Inhibit Bacterial Growth.

The menaquinone analogs, menadione (FIG. 4) and 1,4-naphthoquinone (data not shown), exhibited strong antimicrobial activity against the 4 bacterial species; 1,4-naphthoquinone was approximately 2-fold less active than menadione for all 4 tested organisms.

Menadione was bacteriostatic for S. aureus MN8 at concentrations of 3.1 and 6.25 μg/ml (<3 log drop in CFUs/ml) (FIG. 4A). The compound was bactericidal at concentrations of ≧12.5 μg/ml (≧3 log drop in CFUs/ml; no CFUs/ml were detected after 24 h). Menadione was comparably bacteriostatic and bactericidal for S. aureus strains CDC587 and MNPE at the same concentrations as for strain MN8. Inhibition of TSST-1 production by S. aureus MN8 correlated with inhibition of bacterial growth in that no detectable TSST-1 was produced in the presence of ≧3.1 μg/ml of menadione (lower limit of detection was 1 ng/ml original culture fluid). Doses of <3.1 μg/ml failed to inhibit TSST-1 production compared to untreated S. aureus MN8, which produced approximately 20 μg/ml TSST-1).

Menadione was bacteriostatic for B. anthracis Sterne at concentrations of 6.25 and 12.5 μg/ml and was bactericidal for the organism at ≧25 μg/ml (FIG. 4B). Both Streptococcus pyogenes MNWA (FIG. 4C) and Streptococcus agalactiae MNSI (FIG. 4D) were somewhat more resistant to menadione growth inhibition than S. aureus. Menadione was bacteriostatic for Streptococcus pyogenes MNWA at the menadione concentration of 25 μg/ml (FIG. 4C) and bactericidal (FIG. 4C) at higher concentrations (50 and 100 μg/ml). Menadione was bacteriostatic for Streptococcus agalactiae MNSI at menadione concentrations of 6.25 and 12.5 mg/ml (FIG. 4D) and bactericidal at concentrations of 25, 50, and 100 μg/ml (FIG. 4D).

Coenzyme Q1, 2 and 3 Inhibit Bacterial Growth.

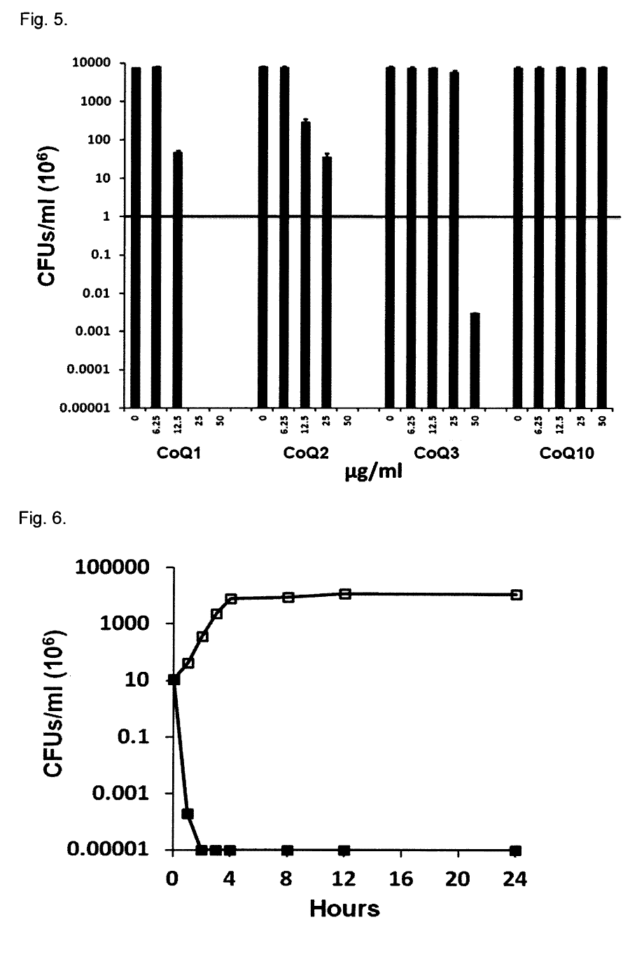

The menaquinone analogs coenzyme Q1-CoQ3, but not coenzyme Q10 were antimicrobial against S. aureus MN8 in the concentration range tested (0-50 μg/ml) (FIG. 5). Coenzyme Q1 was bactericidal (≧3 log reduction in microbial counts) at concentrations of ≧25 μg/ml. Coenzyme Q2 and coenzyme Q3 were bactericidal at a concentration of 50 μg/ml, with coenzyme Q2 having greater reduction in S. aureus counts than coenzyme Q3 at the bacteriostatic concentration of 25 μg/ml. In contrast, coenzyme Q10 exhibited no antimicrobial activity at any concentration tested. TSST-1 production by all three S. aureus strains was completely inhibited by coenzyme Q1-3 at concentrations of 25 and 50 μg/ml, but not at lower coenzyme Q concentrations. Coenzyme Q10 did not inhibit TSST-1 production at any concentration.

A time-course for coenzyme Q1 (50 μg/ml) killing of S. aureus MN8 was performed (FIG. 6). Coenzyme Q1 was bactericidal for MN8 by one hour after inoculation. No microbes were detected after 2 h or any subsequent time-point.

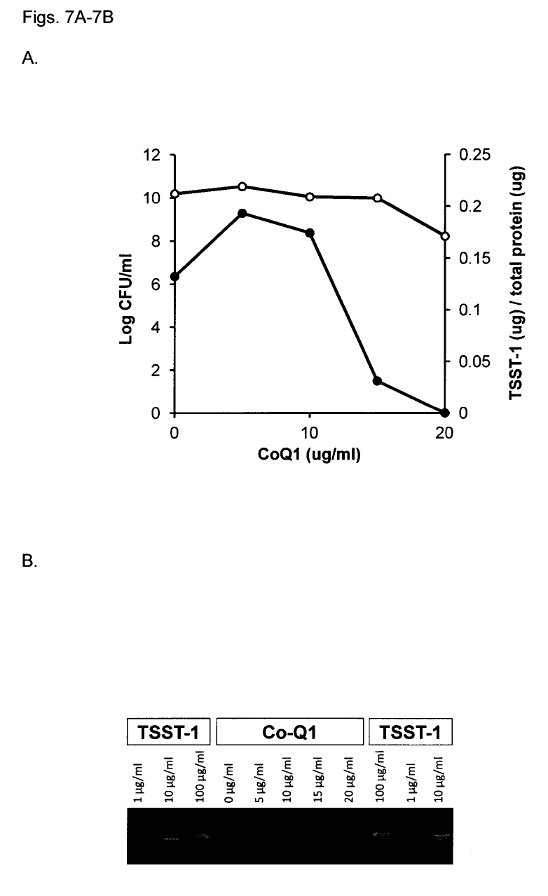

We examined coenzyme Q1 for ability to inhibit exotoxin production (TSST-1) at coenzyme Q concentrations that did not inhibit S. aureus MN8 growth (FIG. 7). S. aureus MN8 was not inhibited from growing over the range of coenzyme Q1 from 0-20 μg/ml (FIG. 7A). In contrast, TSST-1 production was significantly inhibited at coenzyme Q1 concentrations of 10-20 μg/ml (7A,B). There was a 10-fold range over which bacterial growth was not inhibited, yet TSST-1 production was significantly reduced.

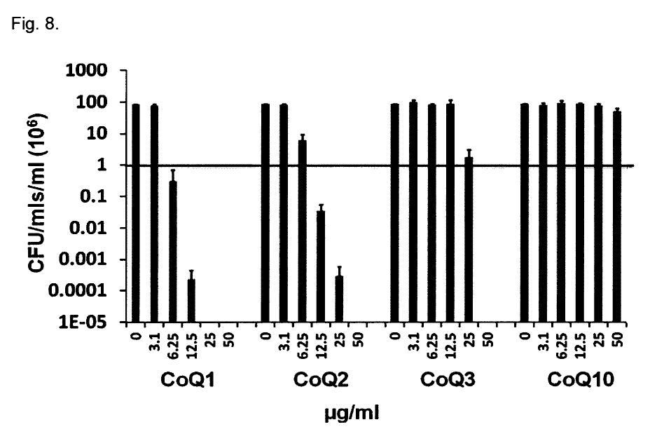

B. anthracis Sterne was killed by coenzyme Q1-3 with greater antimicrobial activity than against S. aureus MN8 (FIG. 8); no antimicrobial activity was observed for coenzyme Q10 against B. anthracis. Coenzyme Q1 was bactericidal for B. anthracis Sterne at coenzyme Q concentrations of ≧12.5 μg/ml. Coenzyme Q1 was bacteriostatic at 6.25 μg/ml. Coenzyme Q2 was bactericidal for B. anthracis at concentrations of ≧25 μg/ml and bacteriostatic at 12.5 μg/ml. Coenzyme Q3 was bactericidal at 50 μg/ml and bacteriostatic at 25 μg/ml.

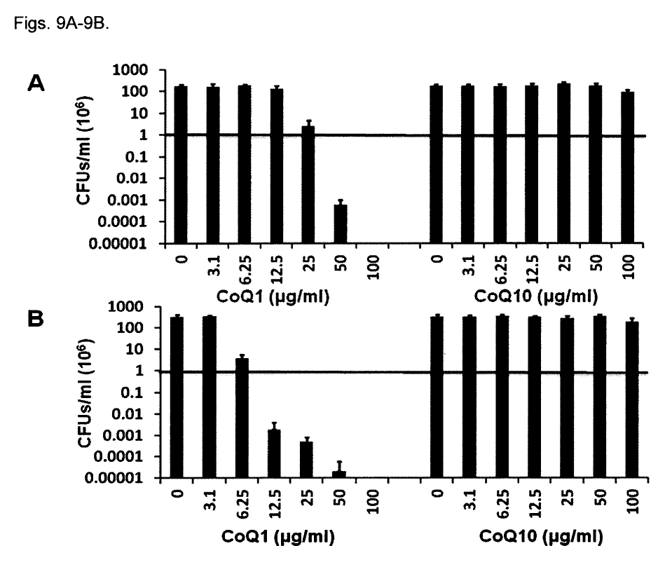

Coenzyme Q1 and coenzyme Q10 were also tested for antimicrobial activity against Streptococcus pyogenes MNWA (FIG. 9A) and Streptococcus agalactiae MNSI (FIG. 9B). Coenzyme Q1 was bactericidal for Streptococcus pyogenes MNWA at concentrations of ≧50 μg/ml and bacteriostatic at 25 μg/ml (FIG. 9A). Coenzyme Q1 was bactericidal for Streptococcus agalactiae MNSI at concentrations of ≧12.5 μg/ml and bacteriostatic at 3.1 and 6.25 μg/ml (FIG. 9B). Coenzyme Q10 lacked antimicrobial activity for either streptococcal strain.

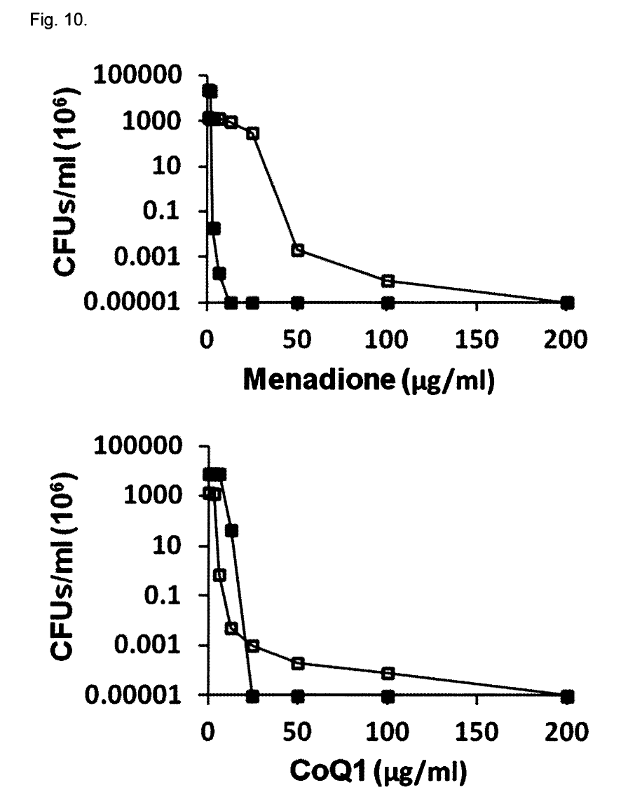

Because studies suggest that menadione may generate toxic reactive oxygen species at concentrations of approximately 10 μg/ml or greater (30), we tested that ability of both menadione and coenzyme Q1 to inhibit growth of S. aureus MN8 (FIG. 10) after culture anaerobically compared to growth aerobically (from data shown in FIGS. 4 and 5). Menadione was bactericidal for S. aureus when grown anaerobically at ≧50 μg/ml, compared to 12.5 μg/ml when cultured aerobically (4-fold difference). However, complete killing of S. aureus MN8 anaerobically did not occur until 200 μg/ml, compared to 12.5 μg/ml aerobically (16-fold difference). Interestingly, coenzyme Q1 was 2-fold more toxic to S. aureus MN8 at low coenzyme Q1 concentrations when cultured anaerobically versus aerobically (though both were bactericidal at 25 μg/ml), but coenzyme Q1 did not completely kill S. aureus MN8 anaerobically until the antimicrobial concentration was 200 μg/ml, compared to 25 μg/ml when cultured aerobically (8-fold difference).

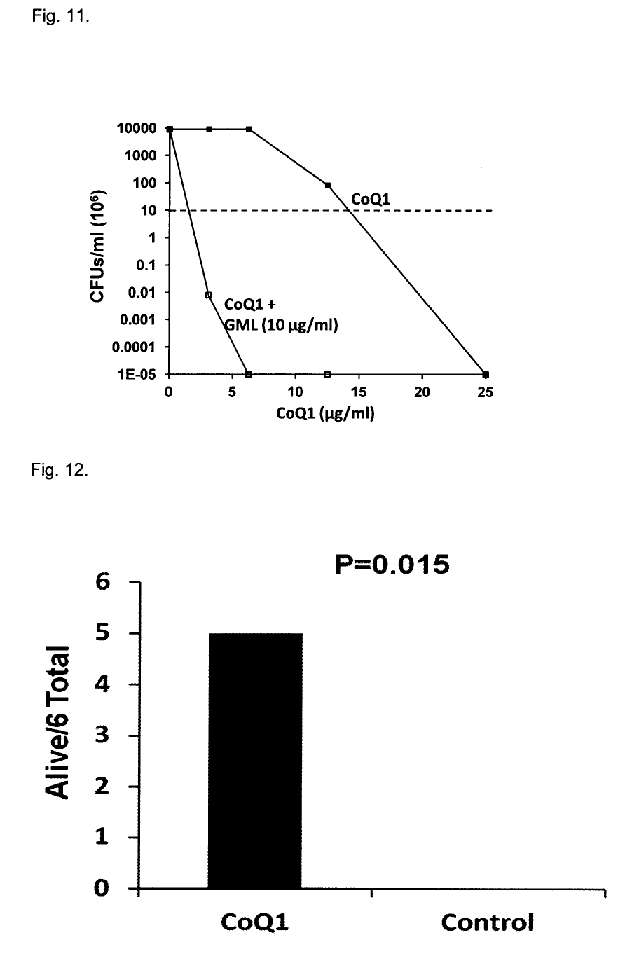

We examined the ability of coenzyme Q1 to synergize with another topical antimicrobial agent, GML, to kill S. aureus MN8 (FIG. 11). Coenzyme Q1 was bactericidal for S. aureus MN8 in this experiment at 15 μg/ml (3 log reduction in CFUs/ml). In contrast, when GML (10 μg/ml) was present, coenzyme Q1 was bactericidal at 2 μg/ml, or a 7.5-fold synergy. GML alone at 10 μg/ml and 100 μg/ml did not inhibit S. aureus MN8 growth.

We also tested coenzyme Q1 for ability to prevent development of TSS in rabbits in a subcutaneous model (FIG. 12). In this model, 5 of 6 rabbits survived and did not develop symptoms of TSS when treated with coenzyme Q1. In contrast all 6 rabbits that received vehicle alone succumbed to lethal TSS induced by S. aureus (P=0.015). There were no gross pathological changes in healthy rabbits treated comparably with coenzyme Q1 but not microbes.

### DISCUSSION

This study addresses the ability of menaquinone analogs to inhibit gram-positive bacterial growth, and independently, exotoxin production. There are several important findings in the study. First, menaquinone analogs, such as menadione, 1,4-naphthoquinone, and coenzyme Q1-3 potently and broadly inhibit gram-positive bacterial growth. Menaquinone is a part of the electron transport chain in S. aureus and B. anthracis (31-33). Menadione is a precursor for menaquinone, so it was surprising that this molecule was highly antimicrobial for these two bacteria. Menadione at concentrations at approximately 10 μg/ml are known to generate reactive oxygen species, including superoxide anions, and this may be an important mechanism of action of the compound (34,35). However, our studies show that menadione is also bactericidal to S. aureus when the organism is grown under anaerobic conditions, suggesting that generation of toxic oxygen radicals is not the only mechanism of menadione antimicrobial activity. The activity of menadione is not as potent under anaerobic conditions as when tested under aerobic conditions. We do not know if coenzyme Q1 molecules generate toxic reactive oxygen species when the cultures are incubated aerobically, but this seems likely. However, it is noteworthy that coenzyme Q1 is more active anaerobically versus aerobically when tested at low concentrations. These data suggest that the major killing mechanism at low concentrations by coenzyme Q1 is not just the generation of toxic reactive oxygen species. Thus, menadione and possibly coenzyme Q1 may exhibit toxicity in part due to superoxide anion generation, but both menadione and coenzyme Q1 must be toxic to bacteria through other mechanisms. We suggest at least one other possibility. In the yeast Saccharomyces cerevisiae, quinones been shown to form covalent bonds with and inactivate macromolecules; this may occur anaerobically as well as aerobically (30). Thus, menadione and coenzyme Q1 may bind to and inactivate key molecules in macromolecular biosynthesis whether on the bacterial cell surface or intracellular.

There have been studies that suggest that menaquinone is required as a signal molecule within the plasma membrane for some two-component systems (21). It is possible that critical two-component systems are negatively affected by menadione and coenzyme Q1 molecules, either through alterations in oxidation-reduction state or through covalent bond formation and inactivation. We recently identified the two-component system SrrA/B (staphylococcal respiratory response) and its homologue BrrA/B (B. anthracis respiratory response) that are related to the Bacillus subtilis ResD/E two-component system (36,37). SrrA/B functions as a repressor of TSST-1 production in the absence of oxygen (37). In the presence of >2% oxygen, SrrA/B is de-repressed and TSST-1 production occurs; this has been proposed to explain the association of tampons with menstrual TSS. The WalK/R system of S. aureus is required for peptidoglycan synthesis, and the two-component system is required for staphylococcal survival (38). If signaling through this system or other key systems is altered by compounds such as menadione and coenzyme Q1 molecules, this could explain their toxicity. Glycerol monolaurate and chitosan malate are membrane-active compounds with broad antimicrobial activity like menadione and coenzyme Q1. We have suggested that these compounds interfere with two-component systems, having demonstrated interference with two-component system function (26). Additionally, we have suggested that glycerol monolaurate, like tetramic acids may dissipate the potential difference across the plasma membrane and in this way interfere with many required bacterial functions (26). We thus suggest that menadione and coenzyme Q molecules may significantly alter the oxidation-reduction state of the plasma membrane and interfere with cell function, either though interference with two-component systems or other required functions of the plasma membrane.

A surprising finding from our studies is that coenzyme Q molecules are broadly antimicrobial, dependent on length of the isoprenoid side chain. Coenzyme Q10 lacks antimicrobial activity, except possibly with minor activity at very high concentrations. Coenzyme Q10 has 10 isoprenoid units in its side chain. In contrast, coenzyme Q1 has the greatest antimicrobial activity, having only one isoprenoid side chain. The activity of coenzyme Q thus must be different from that of glycerol monolaurate mentioned above. Glycerol monolaurate antibacterial activity is greatest with a 12 carbon fatty acid side chain, with reduced activity with shorter fatty acid side chains (26). The data suggest that coenzyme Q antibacterial activity does not simply depend on plasma membrane insertion and interference with membrane fluidity or potential difference dissipation. The present data in fact show that coenzyme Q molecules may synergize with glycerol monolaurate, and thus possibly other antibiotics. Like glycerol monolaurate, in which we have not identified resistant microbes, menadione and coenzyme Q1 in our studies also failed to generate resistant mutants; in all experiments with concentrations of menadione or coenzyme Q1 that kill S. aureus completely, we have not seen resistant colonies arising. These data suggest that menadione and coenzyme Q1 molecules have multiple bacterial targets of antimicrobial activity, with the hypothesis, that if there are many targets, it will be much less likely that resistance will develop.

Another important finding in our studies is that coenzyme Q1 can be administered systemically to inhibit staphylococcal TSS production in a rabbit abscess model. In this model the rabbits administered 500 μg of coenzyme Q1 show no signs of toxicity, and examination grossly of their tissues show no evidence of coenzyme Q1 toxicity. Coenzyme Q1 belongs to the larger family of coenzyme Q molecules, including coenzyme Q10 which is generally recognized as safe (GRAS) by FDA for use as a health food supplement. Indeed, coenzyme Q10 is broadly used to help reduce the incidence of heart diseases, and the molecule is easily tolerated by humans at high doses (grams). It is thus possible that coenzyme Q1-3 with antimicrobial activity will also be tolerated systemically by humans.

We have performed multiple studies of glycerol monolaurate safety and antimicrobial activity in vivo, including both non-human and human chronic safety studies, and human studies of effects on mucosal microbial flora (39-41). Although glycerol monolaurate is also GRAS-listed by FDA as a food supplement, there is little evidence that the molecule can be used systemically. We suggest that coenzyme Q1-3 may be useful as additives to microbicides containing glycerol monolaurate to manage mucosal infections.

In sum, our studies have shown that menadione and coenzyme Q1-3 are broadly antimicrobial for gram-positive bacteria, inhibiting both growth and independently exotoxin production. We have also shown that coenzyme Q1 can be used systemically in rabbits to prevent staphylococcal TSS. It is thus possible that this group of molecules represents a novel class of antimicrobial compounds for managing gram-positive bacterial infections.

### Example 2

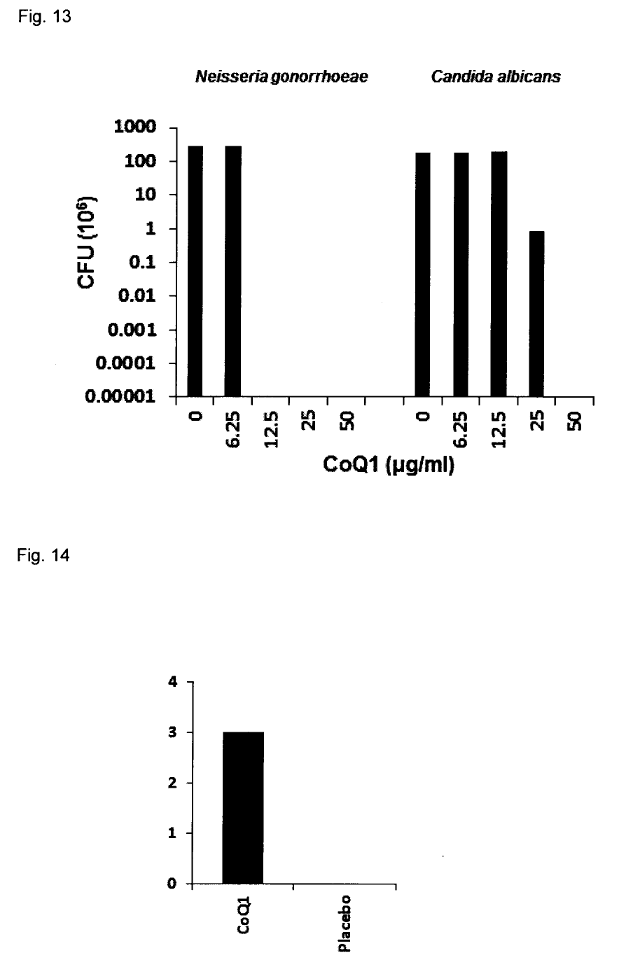

Because CoQ1 was strongly antimicrobial against all gram-positive bacteria tested because CoQ1 is a member of a FDA generally recognized as safe (GRAS) family of compounds, we decided to test the extent of its antimicrobial activity. We showed CoQ1 is bactericidal at 25 and 50 μg/ml for the gram-negative bacterium Gardnerella vaginalis, and the compound was fungicidal for the yeast Candida albicans at 50 μg/ml (FIG. 13).

We also tested CoQ1 for ability to prevent development of TSS in rabbits in a subcutaneous model (FIG. 14). In this model, 3 of 4 rabbits survived and did not develop symptoms of TSS when treated with CoQ1. In contrast all 4 rabbits that received vehicle alone succumbed to lethal TSS induced by S. aureus.

Although the foregoing specification and examples fully disclose and enable the present invention, they are not intended to limit the scope of the invention, which is defined by the claims appended hereto.

All publications, patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### REFERENCES

1. Lowy, F. D. (1998) N Engl J Med 339, 520-532
2. McCormick, J. K., Yarwood, J. M., and Schlievert, P. M. (2001) Annu Rev Microbiol 55, 77-104
3. Dinges, M. M., Orwin, P. M., and Schlievert, P. M. (2000) Clin Microbiol Rev 13, 16-34
4. Schlievert, P. M., Strandberg, K. L., Lin, Y. C., Peterson, M. L., and Leung, D. Y. (2010) J Allergy Clin Immunol 125, 39-49
5. Bergdoll, M. S., Crass, B. A., Reiser, R. F., Robbins, R. N., and Davis, J. P. (1981) Lancet 1, 1017-1021
6. Bergdoll, M. S., and Schlievert, P. M. (1984) Lancet ii, 691
7. Schlievert, P. M., Shands, K. N., Dan, B. B., Schmid, G. P., and Nishimura, R. D. (1981) J Infect Dis 143, 509-516
8. Schlievert, P. M. (1986) Lancet 1, 1149-1150
9. Schlievert, P. M., Tripp, T. J., and Peterson, M. L. (2004) J Clin Microbiol 42, 2875-2876
10. Davis, J. P., Chesney, P. J., Wand, P. J., and LaVenture, M. (1980) N Engl J Med 303, 1429-1435
11. Marrack, P., and Kappler, J. (1990) Science 248, 705-711
12. Shands, K. N., Schmid, G. P., Dan, B. B., Blum, D., Guidotti, R. J., Hargrett, N. T., Anderson, R. L., Hill, D. L., Broome, C. V., Band, J. D., and Fraser, D. W. (1980) N Engl J Med 303, 1436-1442
13. Cone, L. A., Woodard, D. R., Schlievert, P. M., and Tomory, G. S. (1987) N Engl J Med 317, 146-149
14. Schlievert, P. M., Assimacopoulos, A. P., and Cleary, P. P. (1996) J Lab Clin Med 127, 13-22
15. Schlievert, P. M., Gocke, J. E., and Deringer, J. R. (1993) Clin Infect Dis 17, 26-31
16. Stevens, D. L., Tanner, M. H., Winship, J., Swarts, R., Ries, K. M., Schlievert, P. M., and Kaplan, E. (1989) N Engl J Med 321, 1-7
17. Wessels, M. R. N Engl J Med 364, 648-655
18. Wessels, M. R., and Kasper, D. L. (1993) N Engl J Med 328, 1843-1844
19. Koehler, T. M. (2002) Curr Top Microbiol Immunol 271, 143-164
20. Mourez, M., Lacy, D. B., Cunningham, K., Legmann, R., Sellman, B. R., Mogridge, J., and Collier, R. J. (2002) Trends Microbiol 10, 287-293
21. Hoch, J. A., and T. J. Silhavey. (1995) Two-component signal transduction., American Society for Microbiology, Washington, D.C.
22. Novick, R. P. (2003) Mol Microbiol 48, 1429-1449
23. Pragman, A. A., and Schlievert, P. M. (2004) FEMS Immunol Med Microbiol 42, 147-154
24. Schlievert, P. M., Case, L. C., Nemeth, K. A., Davis, C. C., Sun, Y., Qin, W., Wang, F., Brosnahan, A. J., Mleziva, J. A., Peterson, M. L., and Jones, B. E. (2007) Biochemistry 46, 14349-14358
25. Schlievert, P. M., and Kelly, J. A. (1984) J Infect Dis 149, 471
26. Schlievert, P. M., and Peterson, M. L. (2012) PLoS ONE 7, e40350
27. Schlievert, P. M. (2007) Antimicrob Agents Chemother 51, 3056-3062
28. MacDonald, K. L., Osterholm, M. T., Hedberg, C. W., Schrock, C. G., Peterson, G. F., Jentzen, J. M., Leonard, S. A., and Schlievert, P. M. (1987) Jama 257, 1053-1058
29. Yarwood, J. M., McCormick, J. K., Paustian, M. L., Kapur, V., and Schlievert, P. M. (2002) J Bacteriol 184, 1095-1101
30. Rodriguez, C. E., Shinyashiki, M., Froines, J., Yu, R. C., Fukuto, J. M., and Cho, A. K. (2004) Toxicology 201, 185-196
31. Wakeman, C. A., Hammer, N. D., Stauff, D. L., Attia, A. S., Anzaldi, L. L., Dikalov, S. I., Calcutt, M. W., and Skaar, E. P. Mol Microbiol Epub ahead of print
32. Anzaldi, L. L., and Skaar, E. P. Mol Microbiol 82, 1-3
33. Shibata, H., Suzuki, K., and Kobayashi, S. (2007) Can J Microbiol 53, 1091-1100
34. Jamieson, D. J. (1992) J Bacteriol 174, 6678-6681
35. Flattery-O'Brien, J., Collinson, L. P., and Dawes, I. W. (1993) J Gen Microbiol 139, 501-507
36. Vetter, S. M., and Schlievert, P. M. (2007) Biochemistry 46, 7343-7352
37. Yarwood, J. M., McCormick, J. K., and Schlievert, P. M. (2001) J Bacteriol 183, 1113-1123
38. Delaune, A., Poupel, O., Mallet, A., Coic, Y. M., Msadek, T., and Dubrac, S. (2011) PLoS ONE 6, e17054
39. Schlievert, P. M., Strandberg, K. L., Brosnahan, A. J., Peterson, M. L., Pambuccian, S. E., Nephew, K. R., Brunner, K. G., Schultz-Darken, N. J., and Haase, A. T. (2008) Antimicrob Agents Chemother 52, 4448-4454
40. Strandberg, K. L., Peterson, M. L., Lin, Y. C., Pack, M. C., Chase, D. J., and Schlievert, P. M. Antimicrob Agents Chemother 54, 597-601
41. Strandberg, K. L., Peterson, M. L., Schaefers, M. M., Case, L. C., Pack, M. C., Chase, D. J., and Schlievert, P. M. (2009) Clin Infect Dis 49, 1711-1717

1. An antimicrobial composition comprising a non-aqueous carrier and one or more active agents, wherein the active agent is menadione, 1,4-naphthoquinone, Coenzyme Q1, Coenzyme Q2, Coenzyme Q3, Coenzyme Q4, Coenzyme Q5, Coenzyme Q6, Coenzyme Q7, Coenzyme Q8, Coenzyme Q9, and/or Coenzyme Q10, or a pharmaceutically acceptable salt thereof.

2. The antimicrobial composition of claim 1, wherein the active agent is menadione or 1,4-naphthoquinone.

3. The antimicrobial composition of claim 1, wherein the active agent is Coenzyme Q1, Coenzyme Q2, and/or Coenzyme Q3.

4. The antimicrobial composition of claim 1, wherein the non-aqueous carrier is olive oil, vegetable oil, or a petroleum jelly.

5. The antimicrobial composition of claim 1, wherein the active agent is present at a concentration of about 3-500 μg/ml.

6. (canceled)

7. (canceled)

8. The antimicrobial composition of claim 1, further comprising one or more glycerol monoesters (GME).

9. The antimicrobial composition of claim 8, wherein the GME is glycerol linked to a C6-C22 acyl group.

10. The antimicrobial composition of claim 8, wherein the acyl group is branched or unbranched, saturated or unsaturated.

11. The antimicrobial composition of claim 8, wherein the acyl group is derived from a fatty acid.

12. The antimicrobial composition of claim 8, wherein the GME is glycerol monocaprylate, glycerol monocaprate, glycerol monolaurate, or glycerol monomyristate.

13. The antimicrobial composition of claim 8, wherein the GME is glycerol monolaurate (C12).

14. The antimicrobial composition of claim 8, wherein the GME is present at a concentration of about 10-10,000 μg/ml.

15. The antimicrobial composition of claim 1, wherein the composition is at a pH of about 4.0-8.0.

16. The antimicrobial composition of claim 1, further comprising an accelerant.

17. (canceled)

18. An antimicrobial composition comprising propylene glycol (73.55% v/v), polyethylene glycol (25% v/v) and hydroxypropyl cellulose as a gelling agent (1.25% w/v).

19. An apparatus comprising a solid substrate and the antimicrobial composition of claim 1.

20. (canceled)

21. (canceled)

22. The apparatus of claim 19, wherein the solid substrate is a biostable or bioabsorbable composition.

23. A method of inhibiting growth and/or inhibiting production of an exotoxin of a microbe, and/or inhibiting inflammation in an animal in need thereof comprising administering to an animal in need thereof a composition of claim 1.

24. (canceled)

25. (canceled)

26. The method of claim 23, wherein the microbe is a gram-positive bacterium, a gram-negative bacterium, an enveloped virus, or a fungus.

27. The method of claim 26, wherein the microbe is a gram-positive bacterium Staphylococci aureus, Streptococci pyogenes, Streptococci agalactae, or Bacillus anthracis.

28. The method of claim 27, wherein the microbe is Staphylococci aureus and the composition comprises Coenzyme Q1 and GME.

29. The method of claim 26, wherein the microbe is a gram-negative bacterium Gardnerella vaginalis.

30. (canceled)

31. (canceled)

32. (canceled)

33. (canceled)

34. The antimicrobial composition of claim 1, further comprising propylene glycol (73.55% v/v), polyethylene glycol (25% v/v) and hydroxypropyl cellulose as a gelling agent (1.25% w/v).
